(19) United States
(12) Patent Application Publication
(10) Pub. No.: US 2023/0233900 A1
(43) Pub. Date: Jul. 27, 2023

(54) METHOD AND APPARATUS FOR DETERMINING BIOLOGICAL MOVEMENT

(71) Applicant: SHINKO ELECTRIC INDUSTRIES CO., LTD., Nagano (JP)

(72) Inventors: Yuji FURUTA, Nagano (JP); Hisashi KANEDA, Nagano (JP)

(21) Appl. No.: 18/155,357

(22) Filed: Jan. 17, 2023

(30) Foreign Application Priority Data
Jan. 24, 2022 (JP) 2022-008827

(51) Int. Cl. A63B 21/00; A63B 24/00

(52) U.S. Cl. CPC A63B 21/40; A63B 24/0087

(57) ABSTRACT

A method of determining biological movement includes detecting movement of a monitored portion of a person's body with a first sensor, detecting a potential of a muscle responsible for the movement with a second sensor, and determining whether or not the movement is normal, wherein the determining determines that the movement is normal, in response to a joint occurrence of the first sensor detecting the movement and the second sensor detecting the potential of the muscle responsible for the movement.

### CROSS-REFERENCE TO RELATED APPLICATIONS

The present application is based on and claims priority to Japanese Patent Application No. 2022-008827 filed on Jan. 24, 2022, with the Japanese Patent Office, the entire contents of which are incorporated herein by reference.

### FIELD

The disclosures herein relate to methods for determining biological movement and apparatuses for determining biological movement.

### BACKGROUND

Apparatuses and methods for rehabilitating a living body are known in the art. For example, there is a rehabilitation apparatus capable of measuring the degree of grip strength rehabilitation and wrist strength rehabilitation while enabling a single instrument to allow a person to undergo finger strength rehabilitation and wrist strength rehabilitation. This rehabilitation apparatus includes a fixing module fixed to a person's arm, a wrist movement module having a hinge shaft at one end of the fixing module and connected to the fixing module such as to rotate when the person rotates the hand around the wrist, and a grip strength movement module provided in the wrist movement module to measure the person's grip strength.

The above-described rehabilitation apparatus can detect the movement of a person's fingers, but cannot identify the forced movement of a paralyzed finger moved along with the movement of unimpaired fingers when the person cannot move the paralyzed finger. That is, it may not be possible to distinguish whether the monitored portion of a person's body is functioning normally or not functioning normally.

Accordingly, there may be a need to provide a biological movement determination method capable of determining whether the movement of a monitored portion of a person's body is normal or abnormal.

### PRIOR ART DOCUMENT

### Patent Document

Patent Document 1 Japanese Laid-Open Patent Publication No. 2016-97295

### SUMMARY

According to an aspect of the embodiment, a method of determining biological movement includes detecting movement of a monitored portion of a person's body with a first sensor, detecting a potential of a muscle responsible for the movement with a second sensor, and determining whether or not the movement is normal, wherein the determining determines that the movement is normal, in response to a joint occurrence of the first sensor detecting the movement and the second sensor detecting the potential of the muscle responsible for the movement.

The object and advantages of the embodiment will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIG. 1 is a block diagram illustrating a biological movement determination apparatus according to a first embodiment;

FIGS. 2A and 2B are drawings illustrating a first sensor;

FIGS. 3A and 3B are drawings illustrating the functioning of the first sensor;

FIGS. 4A and 4B are drawings illustrating a second sensor;

FIGS. 5A and 5B are drawings illustrating the outputs of the first sensor and the second sensor with respect to a finger that is normally functioning;

FIGS. 6A and 6B are drawings illustrating the outputs of the first sensor and the second sensor with respect to a finger that is not functioning normally;

FIG. 7 is a flowchart illustrating a method of determining biological movement according to the first embodiment;

FIG. 8 is a plan view illustrating a first sensor according to a first variation of the first embodiment; and

FIG. 9 is a flowchart illustrating a method of determining biological movement according to a second variation of the first embodiment.

### DESCRIPTION OF EMBODIMENTS

In the following, embodiments for carrying out the invention will be described with reference to the accompanying drawings. In the drawings, the same components are denoted by the same reference numerals, and a duplicate description thereof may be omitted.

### First Embodiment

### [Configuration of Biological Movement Determination Apparatus]

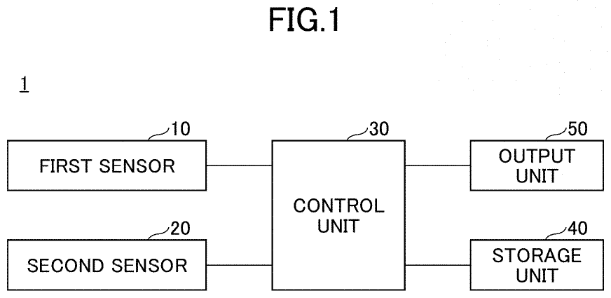

FIG. 1 is a block diagram illustrating a biological movement determination apparatus according to a first embodiment. As illustrated in FIG. 1, the biological movement determination apparatus 1 includes a first sensor 10, a second sensor 20, a control unit 30, a storage unit 40, and an output unit 50. The biological movement determination apparatus 1 may further include an interface or the like that enables the exchange of signals between the control unit 30 and another apparatus.

The first sensor 10 detects the movement of a monitored portion of a person's body. The second sensor 20 detects a myoelectric signal, i.e., the potential of a muscle that controls the movement of the monitored portion of the person's body. The monitored portion of the person's body is, for example, one or more fingers of the person's hand. The detection signal of the first sensor 10 and the detection signal of the second sensor 20 are subjected to predetermined signal processing and then output to the control unit 30. The predetermined signal processing is, for example, noise removal, signal amplification, A/D conversion or the like. The electrical connection between the control unit 30 and either the first sensor 10, the second sensor 20, or both may be a wired connection, or may be a wireless connection. Details of the first sensor 10 and the second sensor 20 will be described later.

The control unit 30 is a processor implemented as an electronic circuit and programmed to execute various functions based on software, an application specific integrated circuit (ASIC) designed to execute a predetermined function, a digital signal processor (DSP), a field programmable gate array (FPGA) , a system on a chip (SOC) , and/or a graphics processing unit (GPU), for example. The control unit 30 determines whether the movement of a monitored portion of a person's body is normal or not normal based on the detection signal of the first sensor 10 and the detection signal of the second sensor 20. The control unit 30 outputs a signal for producing an image, sound, or the like to the output unit 50.

The storage unit 40 is, for example, a read-only memory (ROM), a random-access memory (RAM), a solid-state drive, a hard disk drive, an optical disk drive, or the like. The storage unit 40 may store various programs. The storage unit 40 may temporarily or permanently store data indicative of the detection signals of the first sensor 10 and the second sensor 20.

The output unit 50 is, for example, an image output apparatus such as a liquid crystal display or an organic electro-luminescence (EL) display, a sound output apparatus such as a speaker, or a light-emitting apparatus such as a light-emitting diode indicating pass or fail. The output unit 50 outputs information based on a command from the control unit 30.

The control unit 30, the storage unit 40, and the output unit 50 may be parts of a personal computer, for example.

### [First Sensor 10]

In the disclosed example, the first sensor 10 that detects the movement of a monitored portion of a person's body is a tactile sensor module. In the following description, a finger is taken as an example of the monitored portion of a person's body.

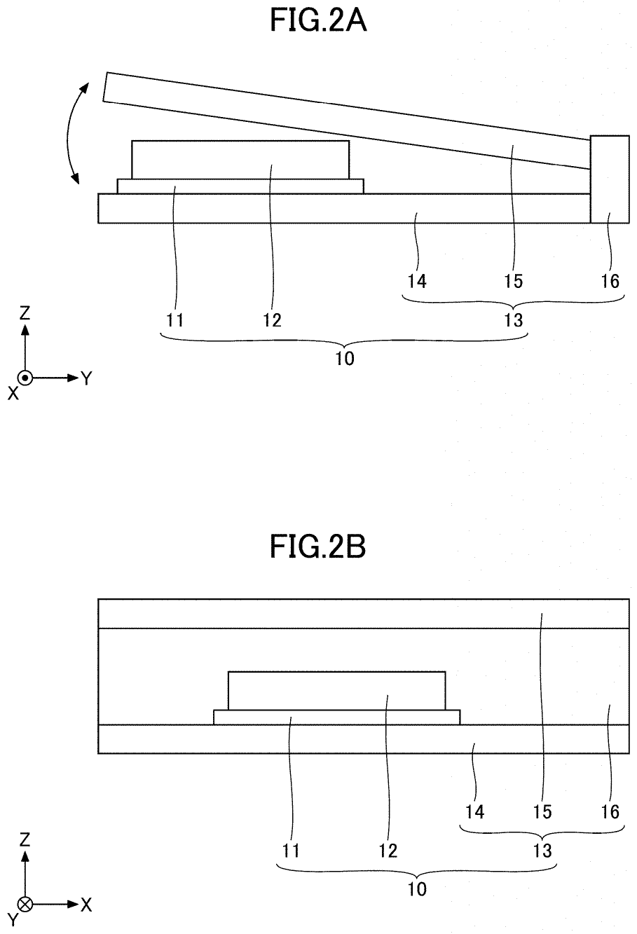

FIG. 2A is a side elevation view of the first sensor, and FIG. 2B is a front view of the first sensor. Referring to FIGS. 2A and 2B, the first sensor 10 is a tactile sensor module having a substrate 11, a tactile sensor 12, and a housing 13. The housing 13 includes a sensor fixing member 14, a movable member 15, and a coupling member 16. The movable member 15 is configured to move in the direction indicated by arrows with the coupling member 16 serving as the axis. The housing 13 may be made of a deformable material such as rubber, for example.

In the housing 13, the tactile sensor 12 mounted on the substrate 11 such as a glass epoxy substrate is disposed on the surface of the sensor fixing member 14 that faces the movable member 15. The tactile sensor 12 is made mainly of a soft material such as sponge, and can detect squeezing of the sponge or the like based on a change in the amount of light passing through the sponge or the like. An example of the tactile sensor 12 is Shokac Cube (registered trademark) manufactured by Touchence Inc.

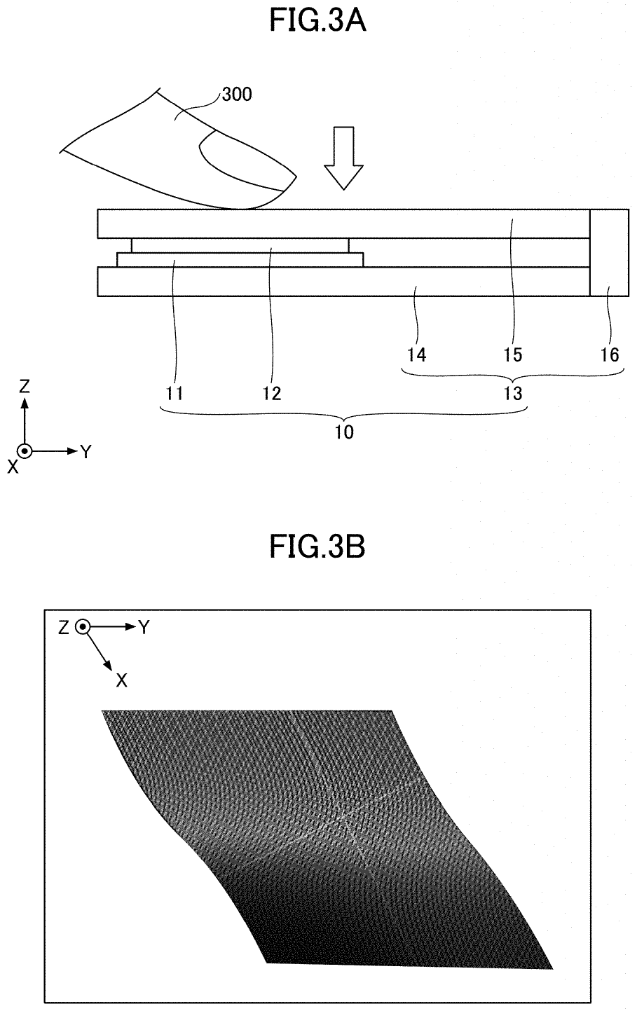

FIGS. 3A and 3B are drawings illustrating the functioning of the first sensor. FIG. 3A is a side elevation view illustrating the movable member 15 of the first sensor 10 that is pressed by a finger 300 to move in a direction indicated by an arrow. Although not illustrated, the finger 300 in this example presses a portion of the tactile sensor 12 on the negative X side of the center of the tactile sensor 12 in the coordinate axis system illustrated in FIG. 2B.

FIG. 3B illustrates the output of the first sensor 10 in the state of FIG. 3A as is displayed on the output unit 50. In FIG. 3B, a portion with a relatively light gray tone is a pressed portion. As can be observed from the displayed image illustrated in FIG. 3B, the finger 300 presses a negative X side of the center of the tactile sensor 12. If the finger 300 pressed a positive X side of the center of the tactile sensor 12 in the coordinate axis system illustrated in FIG. 2B, for example, the gray tone on the positive X side would become lighter in FIG. 3B.

In the manner described above, the first sensor 10 can detect which portion of the tactile sensor 12 is pressed. In the case in which a plurality of fingers are placed at predetermined positions on the sensor fixing member 14 and sequentially exert pressure, it is possible to detect which finger has moved. That is, the first sensor 10 can detect the movement of a plurality of fingers (e.g., five fingers) with one tactile sensor 12.

### [Second Sensor 20]

In the disclosed example, a myoelectric sensor is the second sensor 20 for detecting the potential of a muscle that controls the movement of a monitored portion of a person's body.

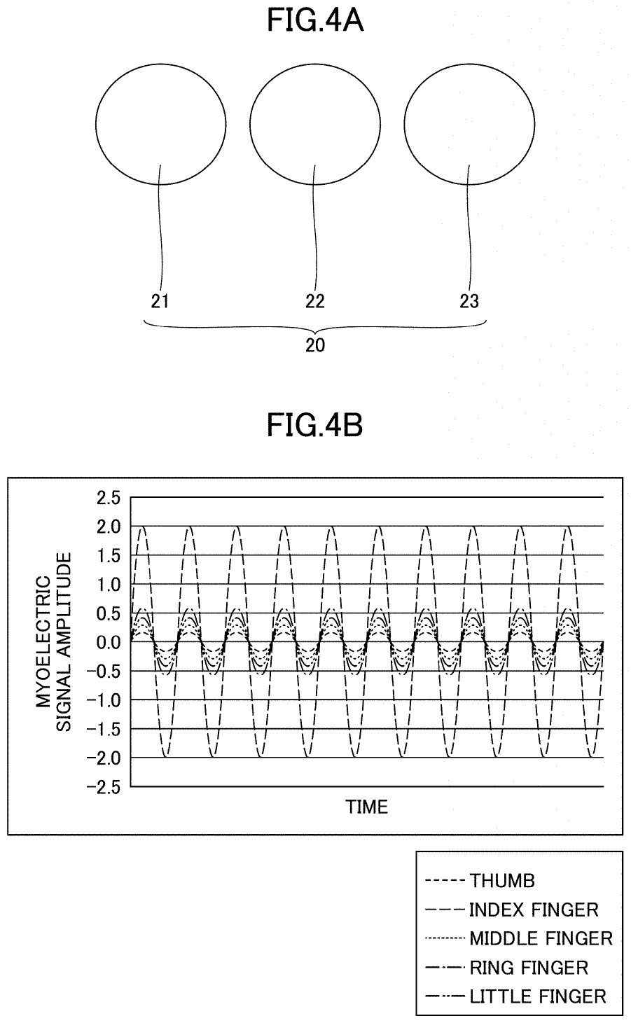

FIGS. 4A and 4B are drawings illustrating the second sensor. FIG. 4A is a schematic drawing illustrating the second sensor 20. Referring to FIG.

4A, the second sensor 20 is a myoelectric sensor having a positive electrode 21, a negative electrode 22, and a reference electrode 23. The reference electrode 23 is provided as needed. The positive electrode 21, the negative electrode 22, and the reference electrode 23 are attached to the surface of the skin of a person's body when used. The positive electrode 21, the negative electrode 22, and the reference electrode 23 detect an electric signal (i.e., myoelectric signal) generated when a muscle for controlling the movement of a monitored portion of a person's body contracts.

The muscles responsible for the movement of fingers are located at different locations in the forearm. It is thus necessary to provide as many second sensors 20 as the number of fingers that are monitored. For example, in the case of detecting the potential of a muscle responsible for the movement of an index finger, the second sensor 20 is arranged at the position of the muscle responsible for the movement of an index finger on the forearm. In the case of detecting the potential of a muscle responsible for the movement of a middle finger, the second sensor 20 is arranged at the position of the muscle responsible for the movement of a middle finger on the forearm. In the case of detecting the potentials of all the muscles responsible for the movement of the five fingers, each second sensor 20 is arranged at the position of the muscle responsible for the movement of a corresponding finger in the forearm.

FIG. 4B schematically illustrates the outputs of the second sensors 20 as displayed on the output unit 50. Specifically, the second sensors 20 are arranged at the positions of respective muscles responsible for the movement of five fingers on the forearm. Signals illustrated here are those observed when only the index finger is moved. As can be seen from FIG. 4B, the output of the second sensor 20 arranged at the position of the muscle responsible for the movement of the index finger on the forearm is greater than the outputs of the other second sensors 20.

### [Operation of Biological Movement Determination Apparatus]

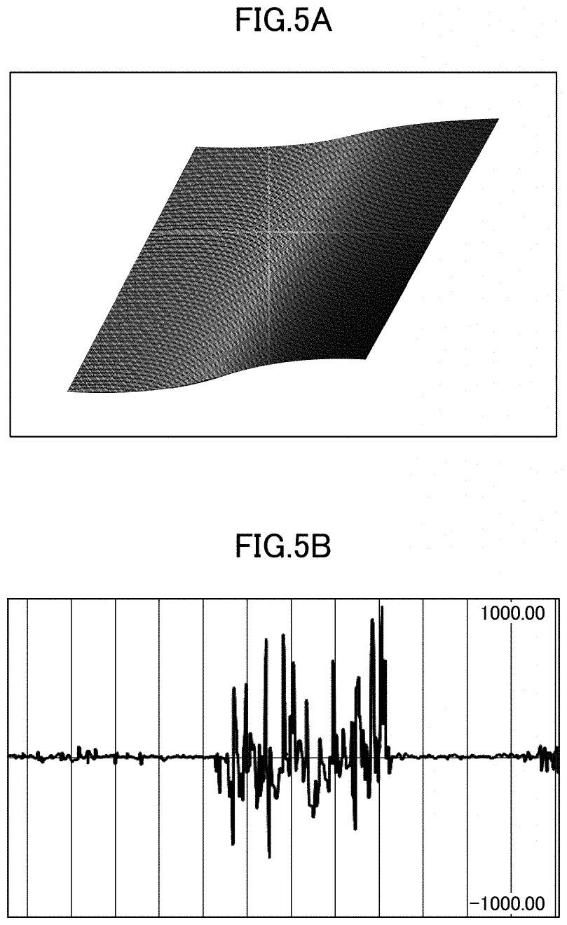

FIGS. 5A and 5B are drawings illustrating the outputs of the first sensor and the second sensor with respect to a finger that is normally functioning. FIG. 5A illustrates the output of the first sensor 10 generated when the middle finger presses this first sensor 10. FIG. 5B illustrates the output of the second sensor 20 arranged at the position of the muscle responsible for the movement of the middle finger when five second sensors 20 are arranged at the respective positions of muscles responsible for the movements of the five fingers on the forearm. FIGS. 5A and 5B indicate that the middle finger is functioning normally.

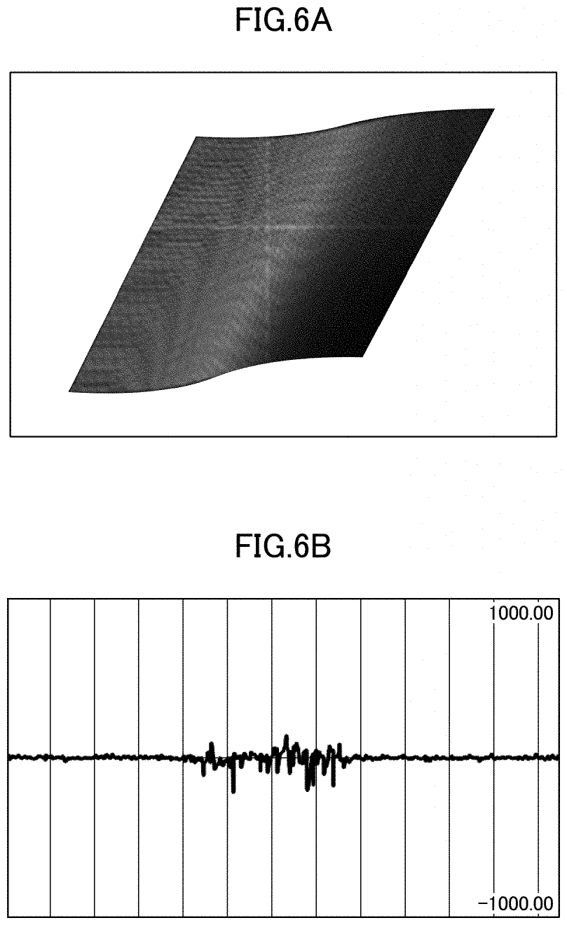

FIGS. 6A and 6B are drawings illustrating the outputs of the first sensor and the second sensor with respect to a finger that is not functioning normally. FIG. 6A illustrates the output of the first sensor 10 in the case in which the middle finger is not functioning normally due to paralysis or the like, but the middle finger is involuntarily moved by the movement of the index finger. FIG. 6B illustrates the output of the second sensor 20 arranged at the position of the muscle responsible for the movement of the middle finger when five second sensors 20 are arranged at the respective positions of muscles responsible for the movements of the five fingers on the forearm. The displayed image illustrated in FIG. 6A is not significantly different from the displayed image illustrated in FIG. 5A. Because of this, such comparison cannot reveal whether the middle finger is functioning normally. On the other hand, the amplitude of the signal illustrated in FIG. 6B is about one fifth of the amplitude of the signal illustrated in FIG. 5B, based on which a determination can be made that the middle finger is not functioning normally.

As described above, when the middle finger is involuntarily moved by the movement of the index finger, observing the myoelectric signal output by the second sensor 20 enables a determination to be made that the middle finger is not functioning normally. The determination as to whether a normal myoelectric signal is detected can be made by detecting whether a change in the potential of a muscle has exceeded a predetermined threshold. Alternatively, the determination may be made based on the integrated value of the myoelectric signal, or may be made by analyzing the frequency spectrum of the myoelectric signal.

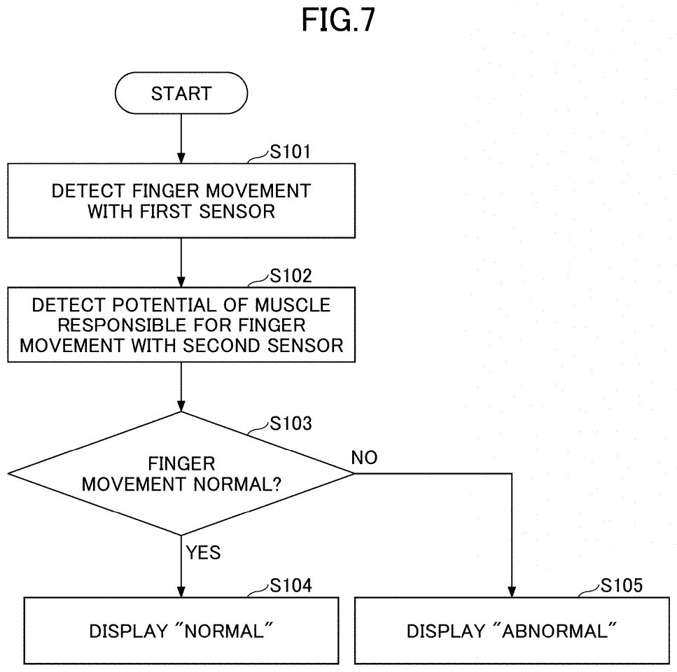

FIG. 7 is a flowchart illustrating a method of determining biological movement according to the first embodiment. In the following description, the method of determining whether or not the movement of a middle finger of a person's body is normal will be used as an example.

In step S101, the second sensor 20 is arranged at the position of a muscle responsible for the movement of a middle finger of a person's body. For example, the second sensor 20 is attached to the position of a muscle responsible for the movement of a middle finger of a person's body. The person is then asked to press the first sensor 10 with the middle finger, and the first sensor 10 detects the movement of the middle finger. The detection signal of the first sensor 10 is transmitted to the control unit 30 and temporarily stored in the storage unit 40, for example.

In step S102, the second sensor 20 detects the potential of the muscles responsible for the movement of the middle finger. The detected output of the first sensor 10 may be used as a trigger to detect the output of the second sensor 20. With this arrangement, detection by the second sensor 20 is made at adequate timing. The detection signal of the second sensor 20 is transmitted to the control unit 30 and temporarily stored in the storage unit 40, for example.

In step S103, the control unit 30 determines whether the movement of the finger is normal. The control unit 30 retrieves relevant information from the storage unit 40, and determines whether the middle finger has moved based on the detection made by the first sensor 10 in step S101. For example, when the signal as illustrated in FIG. 5A is obtained, a determination is made that the middle finger has moved. The control unit 30 further retrieves relevant information from the storage unit 40, and determines whether the potential of a muscle responsible for the movement of the middle finger is detected based on the detection made by the second sensor 20 in step S102. For example, when the signal as illustrated in FIG. 5B is obtained, a determination is made that the potential of a muscle responsible for the movement of the middle finger is detected.

When the first sensor 10 detects the movement of the middle finger and the second sensor 20 detects the potential of a muscle responsible for the movement of the middle finger, the control unit 30 determines that the movement of the middle finger is normal. For example, when the signals as illustrated in FIGS. 5A and 5B are obtained, the control unit 30 determines that the movement of the middle finger is normal. On the other hand, when the first sensor 10 detects the movement of the middle finger but the second sensor 20 fails to detect the potential of a muscle responsible for the movement of the middle finger, the control unit 30 determines that the movement of the middle finger is abnormal. For example, when the signals as illustrated in FIGS. 6A and 6B are obtained, the control unit 30 determines that the movement of the middle finger is abnormal. The method of determining whether the potential of a muscle is detected is as described above. When the first sensor 10 does not detect the movement of the middle finger and the second sensor 20 does not detect the potential of a muscle responsible for the movement of the middle finger, the control unit 30 naturally determines that the movement of the middle finger is abnormal.

In step S103, when the control unit 30 determines that the movement of the middle fingers is normal (YES branch), the process proceeds to step S104. The control unit 30 then issues a command to the output unit 50 such that the output unit 50 displays “normal”, for example. When the control unit 30 determines that the movement of the middle finger is abnormal (NO branch) in step S103, the process proceeds to step S105. The control unit 30 then issues a command to the output unit 50 such that the output unit 50 displays “abnormal”, for example. If necessary, the process may return to step S101 to repeat the monitoring of the same finger, or to monitor all the five fingers sequentially. When “abnormal” is displayed on the output unit, one may choose to cancel subsequent operations.

As described above, the biological movement determination method according to the present embodiment determines whether the movement of a monitored portion of a person's body is normal or abnormal. When finger rehabilitation is performed using the biological movement determination apparatus 1, for example, it is possible to easily determine whether adequate rehabilitation is performed.

In addition, the biological movement determination method according to the present embodiment does not use a large apparatus such as an ultrasonic image diagnostic apparatus or a CT, and utilizes a small, simple configuration combining two types of sensor such as a tactile sensor and a myoelectric sensor to determine whether the movement of a monitored portion of a person's body is normal or abnormal.

Since a myoelectric signal is obtained in the biological movement determination apparatus 1, an assessment of muscle amount can also be made based on data indicative of the amplitude of the obtained myoelectric signal.

### First Variation of First Embodiment

A first variation of the first embodiment is directed to a configuration in which a sensor different than the first embodiment is used. In the first variation of the first embodiment, a description of the same components as those of the above-described embodiment may be omitted.

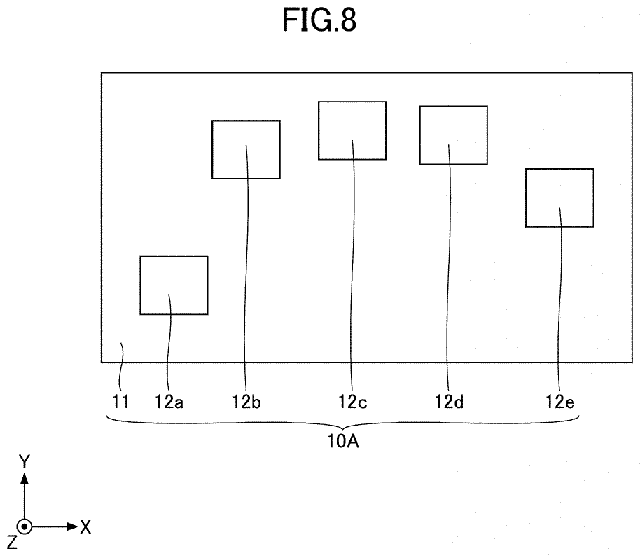

FIG. 8 is a plan view illustrating a first sensor according to a first variation of the first embodiment. The first sensor 10A illustrated in FIG. 8 is a pressure-sensor module including the substrate 11 and the pressure sensors 12a through 12e. A flexible member such as sponge may be provided so as to cover the pressure sensors 12a through 12e. The number of pressure sensors is preferably equal to the number of monitored portions. Examples of the pressure sensors 12a through 12e include a sensor using piezoresistance and a sensor that detects a change in at least one of the following: capacitance; electromagnetism; light; a potential difference; and the like.

In the example illustrated in FIG. 8, the monitored portion is the five fingers of a right hand. The pressure sensor 12a is arranged at such a position as to be easily pressed by the thumb. The pressure sensor 12b is arranged at such a position as to be easily pressed by the index finger. The pressure sensor 12c is arranged at such a position as to be easily pressed by the middle finger. The pressure sensor 12d is arranged at such a position as to be easily pressed by the ring finger. The pressure sensor 12e is arranged at such a position as to be easily pressed by the little finger. In the case of detecting one or more selective fingers, a corresponding number of pressure sensors may be provided. For example, in the case detecting only the movement of a middle finger, the pressure sensor 12c may only be provided on the substrate 11.

As described above, a sensor different from the tactile sensor may alternatively be used as the first sensor 10 as long as the sensor can detect the movement of a monitored portion of a person's body.

Use of a pressure sensor as the second sensor 20 enables not only the measurement of movement made by a monitored portion of a person's body, but also enables the measurement of a pressing force exerted by a monitored portion of a person's body. For example, use of the biological movement determination apparatus 1 for finger rehabilitation enables the evaluation of changes in the pressing force made over time as a result of rehabilitation.

### Second Variation of First Embodiment

A second variation of the first embodiment is directed to a configuration in which data is collected in advance. In the second variation of the first embodiment, a description of the same components as those of the above-described embodiment may be omitted.

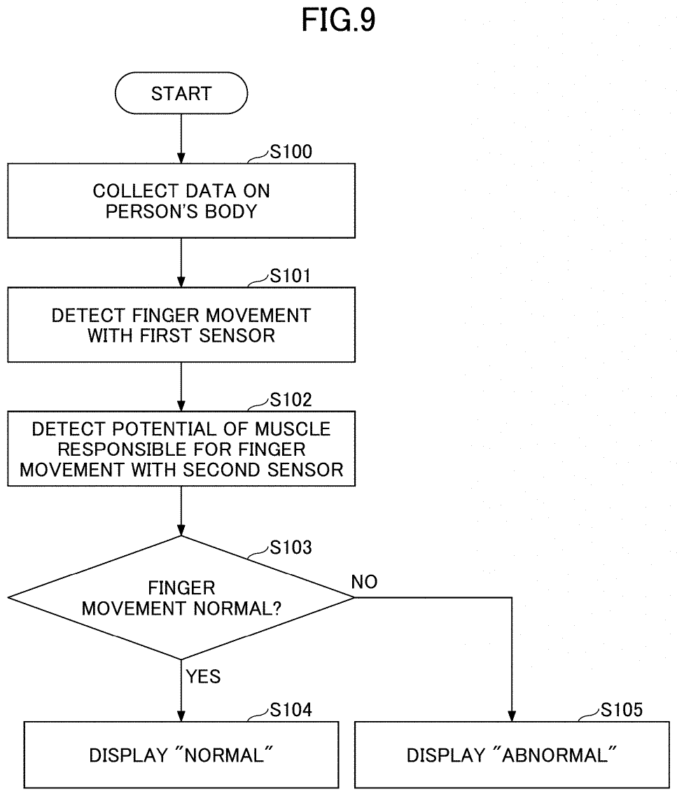

FIG. 9 is a flowchart illustrating a method of determining biological movement according to the second variation of the first embodiment. The flowchart illustrated in FIG. 9 differs from the flowchart illustrated in FIG. 7 in that step S100 is added.

In step S100, data on a person's body is collected. For example, in the case of detecting the movement of a middle finger of a person's body, the second sensor 20 needs to be arranged at the position of a muscle responsible for the movement of the middle finger. The location of a muscle responsible for the movement of a middle finger does not vary significantly from individual to individual. Therefore, the position to attach the second sensor 20 can be determined based on data stored in the database or the like, without collecting data on the person's body in advance as illustrated in the flowchart of FIG. 7.

However, collecting data on the person's body in step S100 allows the second sensor 20 to be arranged at a more precise position. To be more specific, the amplitude of the output of the second sensor 20 obtained upon moving the middle finger is checked by placing the second sensor 20 at a predetermined position. While gradually shifting the position of the second sensor 20 in a stepwise manner, the amplitude of the output of the second sensor 20 obtained upon moving the middle finger is repeatedly checked. Then, the position where the largest amplitude is obtained is determined as the position to attach the second sensor 20. Further, half the largest amplitude may be set as the threshold value. In the case of detecting the movements of a plurality of fingers, data on the person's body can be collected for each finger in the same manner. For the first sensor 10 also, data on the person's body may be collected, and the amplitude of the output of the first sensor 10 (i.e., the density of the displayed tone) or the like obtained upon moving the fingers may be checked.

As described above, by collecting data on a person's body before actually detecting the movement of a monitored portion of the person's body, the detection accuracy of each sensor can be improved.

According to at least one embodiment, it is possible to provide a biological movement determination method capable of determining whether the movement of a monitored portion of a person's body is normal or abnormal.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present inventions have been described in detail, it should be understood that the various changes, substitutions, and alterations could be made hereto without departing from the spirit and scope of the invention.

What is claimed is:

1. A method of determining biological movement, comprising:
detecting movement of a monitored portion of a person's body with a first sensor;
detecting a potential of a muscle responsible for the movement with a second sensor; and
determining whether or not the movement is normal,
wherein the determining determines that the movement is normal, in response to a joint occurrence of the first sensor detecting the movement and the second sensor detecting the potential of the muscle responsible for the movement.

2. The method as claimed in claim 1, wherein the detecting the potential with the second sensor is triggered by an output of the first sensor.

3. The method as claimed in claim 1, wherein the first sensor is a tactile sensor, and the second sensor is a myoelectric sensor.

4. The method as claimed in claim 1, wherein the first sensor is a pressure sensor, and the second sensor is a myoelectric sensor.

5. The method as claimed in claim 1, wherein the monitored portion is one or more fingers of a hand of the person's body,
wherein the first sensor is a single sensor, and as many said second sensors as a number of the one or more fingers are provided,
wherein each of the second sensors is arranged at a position of a muscle responsible for movement of a corresponding one of the one or more fingers on a forearm.

6. The method as claimed in claim 1, wherein the monitored portion is one or more fingers of a hand of the person's body,
wherein as many said first sensors as a number of the one or more fingers are provided, and as many said second sensors as a number of the one or more fingers are provided,
wherein each of the second sensors is arranged at a position of a muscle responsible for movement of a corresponding one of the one or more fingers on a forearm.

7. The method as claimed in claim 1, wherein the determining determines that the movement is normal in response to an occurrence of an event that a part of the monitored portion at which the movement is detected by the first sensor coincides with a part of the monitored portion for which a change in the potential of the muscle detected by the second sensor is greater than or equal to a predetermined threshold.

8. The method as claimed in claim 1, further comprising collecting data on the person's body prior to the detecting the movement with the first sensor.

9. An apparatus for determining biological movement, comprising:
a first sensor configured to detect movement of a monitored portion of a person's body;
a second sensor configured to detect a potential of a muscle responsible for the movement; and
a control unit configured to determine whether the movement is normal,
wherein the control unit determines that the movement is normal in response to a joint occurrence of the first sensor detecting the movement and the second sensor detecting the potential of the muscle responsible for the movement.
